(19) United States
(12) Patent Application Publication
(10) Pub. No.: US 2011/0097746 A1
(43) Pub. Date: Apr. 28, 2011

(54) Orthogonal Method for the Removal of Transmissible Spongiform Encephalopathy Agents from Biological Fluids

(75) Inventors: Jan S. Simoni, Lubbock, TX (US); Grace Simoni, Lubbock, TX (US); John F. Moeller, Lubbock, TX (US)

(73) Assignee: TEXAS TECH UNIVERSITY, Lubbock, TX (US)

(21) Appl. No.: 12/519,163

(22) PCT Filed: Dec. 27, 2007
(86) PCT No.: PCT/US2007/088976

§ 371 Date: Dec. 20, 2010

Related U.S. Application Data
(60) Provisional application No. 60/882,612, filed on Dec. 29, 2006.

(51) Int. Cl. G01N 33/53; B01D 15/42; A61L 2/00; B01D 69/08

(52) U.S. Cl. 435/7.92; 435/7.9; 210/638

(57) ABSTRACT

A method comprising contacting a biological fluid comprising hemoglobin and at least one pathogenic agent with a first filter and generating a first filtrate; contacting the first filtrate with a nanofiltration device and generating a second filtrate; contacting the second filtrate with a chromatographic material and isolating an eluted fraction; contacting the eluted fraction with a hydrophobic solvent and generating a hydrophobic and a hydrophilic phase; and isolating the hydrophilic phase wherein the biological fluids comprise components of interest of equal to or less than about 65 kDa. A method comprising contacting a biological fluid comprising high molecular weight components and at least one pathogenic agent with a first filter and generating a first filtrate; contacting the first filtrate with a hydrophilic membrane and generating a second filtrate; contacting the second filtrate with a chromatographic material and isolating an eluted fraction; contacting the eluted fraction with a hydrophobic solvent and generating a hydrophobic and a hydrophilic phase; and isolating the hydrophilic phase, wherein the high molecular weight components have molecular weights greater than about 65 kDa. A method comprising subjecting a biological fluid comprising hemoglobin and at least one pathogenic agent to at least two filtration steps and thereby reducing the amount of pathogenic agent associated with the biological fluid. A method comprising removing transmissible spongiform encephalopathy agents in a hemoglobin solution of human and/or animal origin by subjecting the hemoglobin solution to an orthogonal separation methodology comprising a plurality of filtration steps.

### FIELD

The present disclosure relates to biological fluids and methods of purifying same. More specifically, this disclosure relates to methods for the orthogonal removal of transmissible spongiform encephalopathy agents from biological fluids.

### BACKGROUND

Transmissible spongiform encephalopathies (TSEs), also known as prion diseases, are a group of rare degenerative brain disorders characterized by tiny holes that give the brain a “spongy” appearance. Creutzfeldt-Jakob disease (CJD) is the most well-known of the human TSEs. It is a rare type of dementia that affects about one in every one million people each year. Other human TSEs include kuru, fatal familial insomnia (FFI), and Gerstmann-Straussler-Scheinker disease (GSS). Kuru was identified in people of an isolated tribe in Papua, New Guinea and has now almost disappeared. FFI and GSS are extremely rare hereditary diseases, found in just a few families around the world. A new type of CJD, called variant CJD (vCJD), was described in 1996 and has been found in Great Britain and several other European countries. The initial symptoms of vCJD are different from those of classic CJD and the disorder typically occurs in younger patients. Symptoms of TSEs vary, but they commonly include personality changes, psychiatric problems such as depression, lack of coordination, and/or an unsteady gait. Patients also may experience involuntary jerking movements called myoclonus, unusual sensations, insomnia, confusion, or memory problems. In the later stages of the disease, patients have severe mental impairment and lose the ability to move or speak. TSEs tend to progress rapidly and usually culminate in death over the course of a few months to a few years. Research suggests that vCJD may have resulted from human consumption of beef from cattle with a TSE disease called bovine spongiform encephalopathy (BSE), also known as “mad cow disease.” Other TSEs found in animals include scrapie, which affects sheep and goats; chronic wasting disease, which affects elk and deer; and transmissible mink encephalopathy. In a few rare cases, TSEs have occurred in other mammals such as zoo animals. There is also evidence to suggest that TSE can be transfusion transmitted however, the time between infection and the appearance of symptoms may be lengthy. For example, humans may be infected for five to twenty years before symptoms appear. Many countries have implemented different measures to prevent TSE outbreaks. The U.S. Food and Drug Administration (FDA) prohibited feeding of ruminants with proteins of animal and implemented a ban on donation from people who have spent more than ten years in France, Portugal and/or Ireland since 1980. People who spent more than six months in Great Britain from 1980-1996 already are forbidden from giving blood in the U.S., Canada, New Zealand, and Australia.

In the United States, the FDA created the TSE Advisory Committee that deals with this subject. Moreover, the FDA has already issued many documents that regulate the presence TSE agent in medicinal products.

Prion diseases such as the TSEs are accompanied by the conversion of normal cellular PrP^{C} into its isoform which are pathogenic prion proteins that are protease-resistant (PrP^{Sc}). PrP^{Sc} are the agents believed responsible for TSE. The risk of contracting a TSE is based on effective exposure of a subject to a TSE agent. Effective exposure is a function of three main variables: the amount of the infectious agent in the contaminated material; the route of exposure; and the specific barrier effect. For example, the parenteral routes of exposure are more efficient in establishing infection than exposure via the alimentary tract. Therefore, current processes for PrP^{Sc} removal, also known as TSE agent removal, are more rigorous for parenteral pharmaceuticals originating in animals and used in humans. Similar measures are also being proposed for pharmaceuticals derived from human tissues.

One challenge to TSE agent removal from blood products comprising hemoglobin is the susceptibility of hemoglobin to degradation. Hemoglobin is a unique and highly unstable molecule that is susceptible to damage during the purification process. This tetrameric heme protein can easily dissociate into unstable dimers and oxidize; therefore losing its ability to transport oxygen, the main purpose of blood substitutes. Spontaneous autoxidation of acellular hemoglobin generates superoxide anion. The rate of this oxidation is augmented by hydrogen ions (low pH). Superoxide anion acts as catalyst and promotes further hemoglobin autoxidation and spontaneously or enzymatically dismutates to form hydrogen peroxide. Hydrogen peroxide reacts with ferrous- or ferric-hemoglobin to produce ferryl-hemoglobin. Ferryl-hemoglobin acts as a radical and initiates lipid peroxidation to the same extent as hydroxyl radicals. The control of hemoglobin oxidative reactions outside of red blood cells is difficult, since this environment does not contain the enzymatic and non-enzymatic antioxidant system that is needed to maintain heme in its functional reduced ferrous form. Thus, irreversible heme oxidation is a problem for hemoglobin-based blood substitute developers.

Hemoglobin solutions, of bovine and human origin, to be effective oxygen carrying plasma expanders, must fulfill a number of requirements. In addition to being non-toxic, non-immunogenic, and non-pyrogenic, having an extended shelf-life, a satisfactory oxygen carrying capacity and colloid osmotic pressure and viscosity similar to plasma; these products should be free of pathogens such as TSE. While the removal of other pathogens from hemoglobin solutions (e.g., microbial) may be effectively achieved using techniques such as sterilization/ultrafiltration followed by a differential culture, the TSE clearance capacity of the manufacturing process must be validated.

Prion proteins (e.g., PrP^{Sc}) are very resistant to common deactivation methods. They can survive cooking and even autoclaving, as well as exposure to a high concentration of acid or base; conditions too aggressive for the purification of fluids comprising hemoglobin. For example, the only pharmaceutical industry method for TSE agent removal in hemoglobin containing solutions is based on a column chromatographic technique.

According to the FDA, a process that is able remove 5 logs of the TSE agent from blood products, particularly hemoglobin solutions, appears acceptable. However, in such a process, log removal by different steps is considered additive only if the clearance steps are orthogonal (i.e., remove the agent by an independent mechanism). Thus, a need exists for an orthogonal method of reducing pathogenic prion proteins from hemoglobin containing solutions.

### SUMMARY

Disclosed herein is a method comprising contacting a biological fluid comprising hemoglobin and at least one pathogenic agent with a first filter and generating a first filtrate; contacting the first filtrate with a nanofiltration device and generating a second filtrate; contacting the second filtrate with a chromatographic material and isolating an eluted fraction; contacting the eluted fraction with a hydrophobic solvent and generating a hydrophobic and a hydrophilic phase; and isolating the hydrophilic phase wherein the biological fluids comprise components of interest of equal to or less than about 65 KDa. Also disclosed herein is a method comprising contacting a biological fluid comprising high molecular weight components and at least one pathogenic agent with a first filter and generating a first filtrate; contacting the first filtrate with a hydrophilic membrane and generating a second filtrate; contacting the second filtrate with a chromatographic material and isolating an eluted fraction; contacting the eluted fraction with a hydrophobic solvent and generating a hydrophobic and a hydrophilic phase; and isolating the hydrophilic phase, wherein the high molecular weight components have molecular weights greater than about 65 kDa. Also disclosed herein is a method comprising subjecting a biological fluid comprising hemoglobin and at least one pathogenic agent to at least two filtration steps and thereby reducing the amount of pathogenic agent associated with the biological fluid. Further disclosed herein is a method comprising removing transmissible spongiform encephalopathy agents in a hemoglobin solution of human and/or animal origin by subjecting the hemoglobin solution to an orthogonal separation methodology comprising a plurality of filtration steps.

### BRIEF DESCRIPTION OF THE DRAWINGS

For a detailed description of the embodiments of the disclosure, reference will now be made to the accompanying drawings in which:

FIG. 1 is a flowchart of a method for reducing the level of TSE agents in a biological fluid.

FIG. 2 is a graphical representation of the effectiveness of an orthogonal multi-step procedure that includes nanofiltration device, ion-exchange membrane chromatography and hydrophobic solvent, in reduction of TSE agent in hemoglobin solution. The results are presented as a Log₁₀ Reduction for individual purification procedures and as a Cumulative Log₁₀ Reduction for the entire multi-step process.

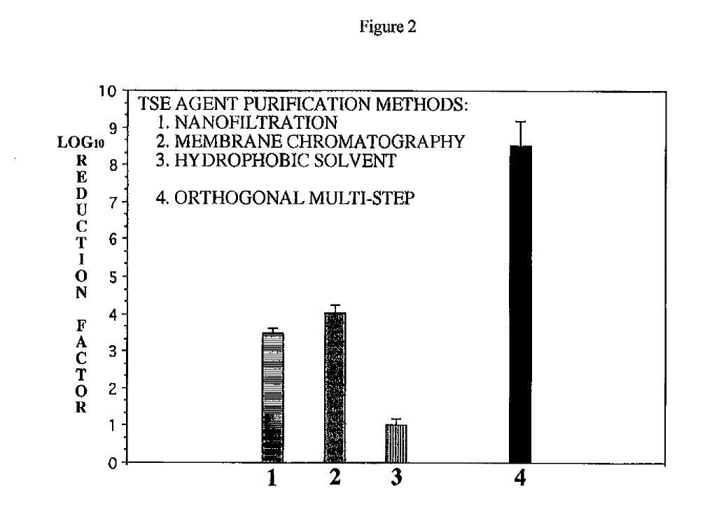

### DETAILED DESCRIPTION

Disclosed herein are methods for the orthogonal removal of pathogenic agents from biological fluids, such as the removal of agents thought responsible for transmissible spongiform encephalaphaties (TSE), hereafter referred to as TSE agents. Herein a biological fluid refers to any fluid having components derived from natural sources, synthetically prepared components, or combinations thereof that may be administered to an organism to treat a disorder. In an embodiment, the biological fluid is a hemoglobin containing solution which may also be referred to as a composition or solution comprising hemoglobin. In an embodiment, the TSE agent is a prion, alternatively a pathogenic prion (PrP^{Sc}). Prion is short for proteinaceous infectious particle and can occur in both a normal form, which is a harmless protein found in the body's cells, and in an infectious form, which causes disease. In an embodiment, the TSE agents may be removed from a hemoglobin containing solution using the orthogonal methodologies disclosed herein, and as used herein the term orthogonal refers to methodologies comprising more than two steps wherein each step results in the removal and/or deactivation of a component (e.g., TSE agent) by independent mechanisms. For example, the orthogonal methodologies described herein may comprise steps that utilize different physiochemical properties of a component (e.g., TSE agent) to effect the removal or elimination of said component.

In an embodiment, the methodology comprises chromatographic techniques, chemical treatment, and nanofiltration in order to effect TSE agent removal from the biological fluid. For example, an orthogonal multi-step procedure may include a high affinity prion reduction filter, a nanofiltration device, a hydrophilic membrane, ion-exchange membrane chromatography and a hydrophobic solvent. In an embodiment, the methodologies for TSE agent removal may be carried out in any order desired by the user, alternatively the methodologies for TSE agent removal may be carried out in the sequence disclosed herein. The resultant biological fluid having been subjected to TSE agent removal (i.e., PrP^{sc} removal) may be suitable for use in the treatment of mammalian disorders requiring the administration of a biological fluid such as a hemglobin containing solution.

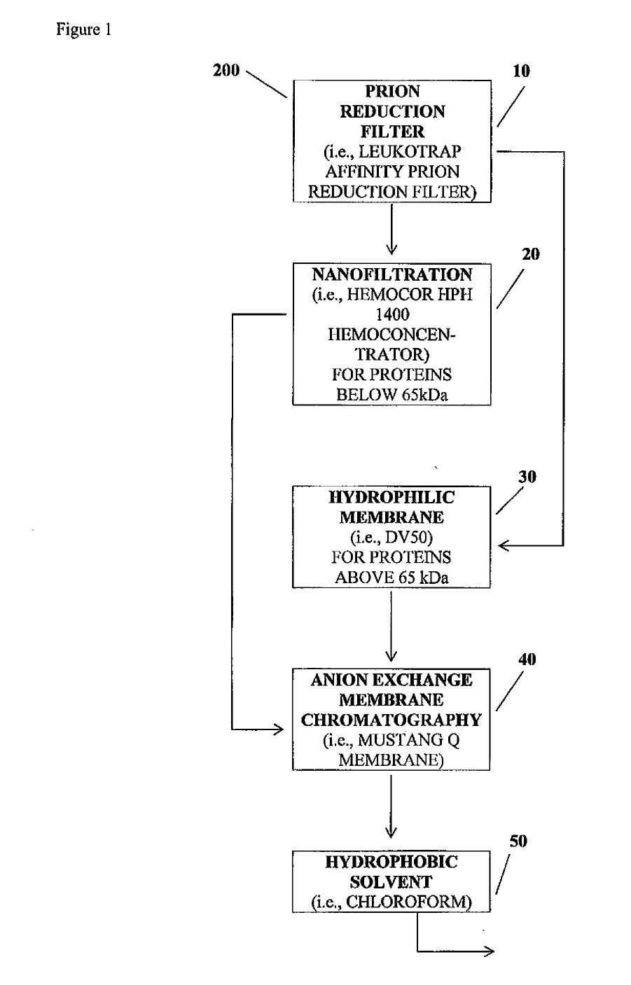

An embodiment of a method for TSE agent removal from a sample, 200, is set forth in FIG. 1. In an embodiment the sample comprises a biological fluid such as a hemoglobin containing solution. The hemoglobin containing solution may comprise hemoglobins of human and animal (e.g., ruminant such as bovine) origin. In an embodiment, the hemoglobin containing solution is derived from whole blood and is an acellular hemoglobin containing solution. The acellular hemoglobin containing solution as used hereinafter may be at a pH that is about the pI (isoelectric point) of hemoglobin, alternatively from about 6.6 to about 7.2, alternatively from about 7.8 to about 8.2 unless otherwise indicated. Prior to being subjected to the methodologies disclosed herein the solution may be contacted with carbon monoxide so as to convert the free hemoglobin to the carbon monoxy form. The carbon monoxy form refers to hemoglobin bound to carbon monoxide. The sample may be contacted with carbon monoxide for a time period sufficient to saturate the sample with carbon monoxide. As will be understood by one of ordinary skill in the art, the time period required to achieve a saturating amount of carbon monoxide will depend on a variety of factors such as the components of the sample solution and the carbon monoxide source and may be adjusted to achieve a user-desired result. Without wishing to be limited by theory, the carbon monoxy form of hemoglobin may be more stable relative to deoxyhemoglobin (i.e. hemoglobin not bound to oxygen) or oxyhemoglobin (i.e., hemoglobin bound to oxygen). In an embodiment, the sample is a biological fluid comprising carbon monoxy hemoglobin. Such samples may be subjected to a methodology as described in blocks 10, 20, 40 and 50. In an embodiment, the final composition obtained after being subjected to the disclosed methodologies has components of interest (i.e. user-desired components) having a molecular weight of less than about 65 kDa.

Referring to FIG. 1, the method 200 may initiate with contacting the sample with a high flow affinity prion reduction filter, block 10. Such high flow affinity prion reduction filters may be comprised of one or more platelet-reducing and/or leukocyte-reducing agents coupled to an inert membrane comprising for example of polymeric materials such as polybutylene terephthalate (PBT), polyethylene, polyethylene terephthalate (PET) and the like. The filter may allow for the rapid flow of fluids (i.e., high flow), such as for example and without limitation biological fluids, at a rate of from about 500 to about 1000 mL of fluid in equal to or less than about 25 minutes, alternatively, in equal to or less than about 20 minutes. Such filters are described in U.S. Pat. No. 6,945,411, which is incorporated by reference herein in its entirety. An example of suitable high flow affinity prion reduction filter is PALL LEUKOTRAP AFFINITY PRION REDUCTION FILTRATION SYSTEM; a whole blood collection, filtration and storage system, commercially available from Pall Corporation (Ann Arbor, Mich. 48103-9019, U.S.A.).

Without wishing to be limited by theory, the high flow affinity prion reduction filter may function to selectively remove PrP^{Sc}-containing leukocytes. Accordingly, block 10 provides a reduction in TSE agents associated with leukocytes, and the filter may be sized accordingly to trap such infected leukocytes. Such filtration may be referred to as leukofiltration.

Extraction of hemoglobin from red blood cells to obtain the starting material which is acellular hemoglobin is typically performed using techniques that damage cellular components. For example, the extraction of hemoglobin from a red blood cell suspension may be carried out by hypo-osmotic lysis. Hypo-osmotic lysis may rupture leukocytes containing PrP^{Sc} and thus releasing TSE agents (i.e., PrP^{Sc}) into the hemoglobin containing solution. Leukofiltration, or the process of removing leukocytes by filtration (e.g., using a high flow prion affinity filter) will decrease the possibility of transferring PrP^{Sc} from leukocytes to free hemoglobin solutions.

Contacting of the sample with the high flow affinity prion reduction filter may result in the removal of equal to or greater than about 1 log of TSE agent from the sample, (e.g., hemoglobin containing solution), alternatively from about 40 to about 60% (0.4-0.6 logs reduction), alternatively from about 0.7 to about 1.9 logs, alternatively from about 2 to about 3.7 logs as determined by a bioassay and a Western blot assay. A sample (e.g., hemoglobin containing solution) after having been subjected to a high flow prion affinity reduction filter is hereinafter termed a filtered sample. The filtered sample comprises the filtrate or the portion of the sample that was not retained by the high flow prion affinity reduction filter.

Referring again to FIG. 1, the method 200, may then proceed to block 20 and the filtered sample contacted with a second filtration device. The potential effectiveness of filtration as a means of TSE removal is based on the fact that the TSE agent (i.e., PrP^{Sc}) can exist in the form of an unusual filamentous morphology with a mass of up to about 1000 kDa. The second filtration device may comprise a nanofiltration device such as for example a hollow fiber filter or disc comprising a porous size-selective membrane. Such nanofiltration devices may be comprised of polymeric materials such as cellulose acetate, cellulose diacetate, cellulose triacetate, polysulfone and the like. The filter may allow for the rapid flow of fluids, such as for example and without limitation biological fluids, at a rate of about 100 mL to about 500 mL of fluid per minute. In an embodiment, the second filtration device has a molecular weight cutoff (meaning the molecules having a molecular weight of equal to or greater than the specified amount are trapped by the filter and molecules having a smaller molecular weight are not retained by the filter) of about 64.5 kDa, alternatively about 65 kDa, alternatively about 75 kDa. In an embodiment, the filter has a size cutoff just slightly larger than a hemoglobin molecule (e.g., 64.5 kDa) such that hemoglobin is not retained by the filter but larger molecules such as TSE agents (e.g., pathogenic prions) are trapped by the filter. Examples of suitable nanofiltration devices include without limitation HEMOCOR High Performance Hemoconcentrator HPH 400, HPH 700, HPH 1000 or HPH 1400, commercially available from Minntech Corporation, Minneapolis, Minn. 55447, U.S.A.; that can be used as a single filtration unit or in a coupled manner to increase the filtration area. In an embodiment, these nanofiltration devices result in a further reduction of TSE agents with molecular mass of equal to or than about 65 kDa, alternatively equal to or greater than about 75 kDa. The filtered sample having been subjected to a second filtration device may have a reduction of equal to or greater than about 1 log, alternatively from about 1 to about 3.2 logs, alternatively of from about 3.3 to about 3.7 logs, alternatively of from about 3.8 to about 4.5 logs in the amount of TSE agent when compared to the filtered sample and is referred to or termed a sized filtered sample. The sized filtered sample comprises the filtrate or the material from filtered sample that was not retained by the filtration device.

In an embodiment, samples such as those described herein which have been subjected to filtration devices may be diluted with respect to the original biological fluid. Dilute samples may be inconvenient to handle as they may comprise a large volume of liquid. Further many biological components (e.g. hemoglobin, proteins, etc . . . ) may display a reduced stability when maintained at low concentrations in a dilute solution. In an embodiment, the solutions generated by the methodologies disclosed herein may be concentrated following a particular technique to generate a more concentrated sample. Suitable techniques for concentrating these samples are known. For example, the sample may be concentrated following contacting with a nanofiltration device by introducing the sample to a dialyzer having a molecular cutoff of about 10 kDa, alternatively about 40 kDa, alternatively about 50 kDa, to concentrate the filtered sample. Alternatively, the biological fluid may be concentrated following each step in the disclosed methodology. The starting concentration and final concentration of the sample will depend on the type of device utilized. Consequently, the final concentration of the sample may be adjusted to a user-desired value by one of ordinary skill in the art.

Referring again to FIG. 1, the method for reduction of TSE agents in a sample may then proceed to block 40 and the sized filtered sample contacted with a chromatographic material or membrane, for example an ion-exchange membrane. In an embodiment, the chromatographic membrane functions to further reduce the level of TSE agents (e.g., PrP^{Sc}) in the sample. In an embodiment, the chromatographic membrane comprises a strong anion exchanger. In alternative embodiments, the chromatography material comprises an anion exchange disc, alternatively an anion exchange capsule, alternatively an anion exchange module. Examples of chromatographic materials suitable for use in this disclosure include without limitation MUSTANG Q Strong Anion Exchange Membrane in the form of ASTRODISC CHROMATOGRAPHY UNIT, MUSTANG Q DISPOSABLE CAPSULE, and MUSTANG Q MODULE; with a porosity of about 0.8 μm and a membrane bed volume from about 0.18 mL to about 1000 mL, alternatively greater than about 1000 mL. MUSTANG Q membranes are commercially available from Pall Corporation. (Ann Arbor, Mich. 48103-9019, U.S.A.). The use of a membrane comprising the MUSTANG Q strong anion exchanger may provide the advantages of desirable low protein-binding properties, broad chemical and temperature resistance, and high flow rate. For example, a modified MUSTANG Q membrane may reduce the level of TSE agents while allowing for transmission of a high percentage of proteins such as for example hemoglobin. The sized filtered sample having been contacted with a chromatographic membrane may have a reduction of equal to or greater than about 1 log, alternatively from about 3.8 to about 4.3 logs, alternatively of from about 1 to about 3.7 logs, alternatively of from about 4.3 to about 5 logs in the amount of TSE agent when compared to the filtered sample and is hereinafter termed a chromatographed sized filtered sample. The chromatographed sized filtered sample comprises an eluted fraction of the composition such that sample comprises material that did not adhere to the anion exchanger.

Referring again to FIG. 1, the method may then proceed to block 50 and the chromatographed sized filtered sample contacted with a hydrophobic solvent. Prior to contact with the hydrophobic solvent, the pH of the sample may be increased to about 8.0, alternatively about 7.8, alternatively about 8.2. Without wishing to be limited by theory, increasing the pH of the chromatographed sized filtered sample (i.e. comprising hemoglobin) will deprotonate the hemoglobin molecule resulting in a negatively charged molecule and facilitate partitioning of the hemoglobin into the hydrophilic phase. In an embodiment, the chromatographed sized filtered sample is contacted with a hydrophobic solvent, agitated, and subsequently allowed to form at least two phases (e.g. hydrophobic and hydrophilic phase) such that at least one component of the sample becomes associated with the hydrophobic phase and at least one component of the sample remains associated with the hydrophilic phase. The hydrophobic solvent may be any hydrophobic solvent that is compatible with the components of the chromatographed processed sample; alternatively the hydrophobic solvent comprises chloroform, toluene, or combinations thereof. Without wishing to be limited by theory, the aggregated forms of the TSE agent (e.g., PrP^{Sc}) may have increased solubility in a hydrophobic solvent and thus may preferentially partition into the hydrophobic solvent further reducing the amount present in the sample. Further, partitioning of the TSE agent into the hydrophobic solvent may result in degradation of the TSE agent. Thus, contacting of the biological fluid with a hydrophobic solvent reduces the presence and infectivity of the TSE agent. In an embodiment, block 50 may further comprise subjecting the chromatographed processed sample that was contacted with the hydrophobic solvent to centrifugation, alternatively high-speed ultracentrifugation. Centrifugation may be employed in order to facilitate the partitioning of the chromatographed processed sample into a hydrophobic and a hydrophilic phase. Methods and equipment for the separation of a sample using techniques such as centrifugation are known to one of ordinary skill in the art. In an embodiment, the hydrophilic phase of the chromatographed sized filtered sample that may then be employed in the subsequent steps of the method disclosed herein may have a reduction of equal to or greater than about 1 log, alternatively from about 0.8 to about 1.2 logs, alternatively of from about 0.1 to about 0.7 logs, alternatively of from about 1.3 to about 3.5 logs in the amount of TSE agent when compared to the chromatographed processed sample and is referred to or termed the processed sample.

In an embodiment, the method may then allow for further processing of the processed sample to place the sample in a condition suitable for introduction to an organism such as for example, administration to a patient. Alternatively, the sample, (e.g., hemoglobin of human or animal origin) may be used with further processing in the manufacturing of free hemoglobin based blood substitutes.

In an alternative embodiment, the biological fluid comprises plasma or serum. Plasma samples may comprise its fractions such as albumin, clotting factors, immunoglobulins or combinations thereof. Such samples may be subjected to a methodology as described in blocks 10, 30, 40 and 50. In an embodiment, the final composition to be obtained after subjecting the plasma or serum to the disclosed methodologies have components of interest (i.e. user-desired components) having a molecular weight of greater than about 65 kDa and equal to or less than about 150 kDa.

Referring to FIG. 1, a method of reducing the level of TSE agents in the sample may begin at block 10 and comprise a high flow affinity prion reduction system suitable for use with biological fluids having high molecular weight components such as immunoglobulin (150 kDa). Herein high molecular weight refers to molecular weights of greater than about 65 kDa and such biological fluids comprising said high molecular weight components are termed high molecular weight samples (HMWS). An example of a high flow prion reduction filter suitable for use in the removal of TSE agents from a HMWS includes without limitation LEUKOTRAP SC RC Filtration System which is commercially available from Pall Corporation. The isolation of red blood cells, platelets and leukocyte from these HMWS may require invasive techniques such as centrifugal forces that can damage PrP^{Sc} containing leukocytes and may introduce the TSE agent (i.e., PrP^{Sc}) into the sample. A HMWS when contacted with a high flow prion reduction filter of the type described herein may have the components of interest remain in solution (e.g., IgG) while TSE agents are trapped by the filter. The solution that is removed from the filter contains the components of interest that may be subsequently processed and the sample is hereinafter termed a filtered HMWS. The filtered HMWS may have a reduction in the amount of TSE agent of equal to or greater than about 1 log, alternatively of from about 0.7 to about 1.9 logs, alternatively from about 2 to about 3.7 logs when compared to the HMSW. Referring to FIG. 1, the method may then proceed to block 30 and the filtered HMWS may be contacted with a hydrophilic membrane.

The hydrophilic membrane may function to further reduce the level of TSE agents (e.g., PrP^{Sc}) in the HMWS. In an embodiment, the membrane comprises polyvinylidene fluoride (PVDF), alternatively modified PVDF. The use of a membrane comprising PVDF may provide the advantages of desirable low protein-binding properties, broad chemical and temperature resistance, and high flow rate. For example, a modified PVDF membrane may reduce the level of TSE agents while allowing for transmission of a high percentage of proteins such as for example hemoglobin. An example of a hydrophilic PVDF membrane suitable for use in this disclosure includes without limitation ULTIPOR Grade DV50 membrane filter commercially available from Pall Corporation. Examples of suitable PVDF membranes are disclosed in U.S. Pat. No. 5,736,051, which is incorporated by reference herein in its entirety. A filtered HMWS sample that has been contacted with a hydrophilic membrane, hereinafter termed a processed HMWS, may have a reduction of equal to or greater than about 1 log, alternatively of from about 3.3 to about 3.7 logs, alternatively of from about 1 to about 3.2 logs, alternatively of from about 3.8 to about 4.5 logs in the amount of TSE agent when compared to the filtered HMWS. The filtrate from the hydrophilic membrane may then be employed in the subsequent steps (e.g., blocks 40 and/or 50) of the method disclosed herein.

In an embodiment, the processed HMSW is then contacted with an anion exchanger (e.g., block 40) and subsequently a hydrophobic solvent (e.g., block 50) as was described previously herein for a hemoglobin containing solution. Following contacting of the HMSW with an anion exchanger (e.g., block 40) the sample may have a reduction in the amount of TSE agent of equal to or greater than about 1 log, alternatively of from about 3.8 to about 4.3 logs, alternatively from about 1 to about 3.7 logs, alternatively from about 4.3 to about 5 logs when compared to the HMSW not subjected to the anion exchanger. Following contacting of the HMSW with a hydrophobic solvent (e.g., block 50), the sample may have a reduction in the amount of TSE agent of equal to or greater than about 1 log, alternatively of from about 0.8 to about 1.2 logs, alternatively from about 0.1 to about 0.7 logs, alternatively from about 1.3 to about 3.5 logs when compared to the HMSW not subjected to the hydrophobic solvent. As described previously, the method may then allow for further processing of the processed sample to place the sample in a condition suitable for introduction to an organism such as for example, administration to a patient. Alternatively, the sample may be used without further processing.

In an embodiment, the method further comprises determining the level of TSE agent in the samples prior to, during, or after the sample has been subjected to the disclosed methodologies. For example, at least a portion of the sample may be analyzed for the presence of TSE agents (e.g. PrP^{Sc}) prior to contacting the sample with a nanofiltration device, FIG. 1 block 20. Alternatively, at least a portion of the sample may be analyzed for the presence of TSE agents following contacting the sample with an anion exchange membrane, FIG. 1 block 40. Alternatively, at least a portion of the sample may be analyzed for the presence of TSE agents following contacting the sample with a hydrophobic solvent, FIG. 1 block 50. In some embodiments, the method further comprises analyzing at least a portion of the sample for the presence of TSE agents following each step in the disclosed methodology. Analysis for the presence of TSE agents may be qualitative, quantitative or both. Such analyses are known to one of ordinary skill in the art and may include for example Western blots, ELISA, animal infectivity assays or combinations thereof. In an embodiment, a sample having been subjected to the TSE agent removal processes disclosed herein (e.g., partitioned chromatographed processed sample) may have a removal of equal to or greater than about 5 logs of the TSE agents present in the sample, alternatively equal to or greater than about 6 logs, alternatively equal to or greater than about 7 logs. In an embodiment, the sample having been subjected to the methodologies disclosed herein may have undetectable levels of TSE agents wherein the, methods for detection comprise ELISA, animal infectivity assays or combinations thereof. A sample comprising infectious amounts of one or more TSE agents when subjected to the methodologies disclosed herein may have a sufficient reduction in the amount of TSE agents present to result in the loss of the infectivity of the sample.

The methods described herein may be carried out manually, may be automated, or may be combinations of manual and automated processes. In an embodiment, devices for the implementation of the methodologies described herein may be controlled manually, may be automated or combinations thereof. In an embodiment, the method is implemented via a computerized apparatus capable of performing the processes disclosed herein, wherein the method described herein is implemented in software on a general purpose computer or other computerized component having a processor, user interface, microprocessor, memory, and other associated hardware and operating software. The software implementing the method may be stored in tangible media and/or may be resident in memory, for example, on a computer. Likewise, input and/or output from the software, for example component amounts, comparisons, and results, may be stored in a tangible media, computer memory, hardcopy such as a paper printout, or other storage device.

The methodologies disclosed herein are a PrP^{Sc} clearance platform that comprises individual elimination steps that depend on different physical principles and address typical properties of PrP^{Sc}. The methodologies disclosed herein comprise PrP^{Sc} reduction by removal of leukocytes; PrP^{Sc} filtration with nanofilters, PrP^{Sc} absorption with anionic membrane absorbents and PrP^{Sc} inactivation with hydrophobic solvent.

### EXAMPLES

This embodiments having been generally described, the following examples are given as particular embodiments and to demonstrate the practice and advantages thereof.

It is to be understood that the examples are given by way of illustration and are not intended to limit the specification of the claims in any manner.

### Example One

### Purification of Bovine Hemoglobin Solution by Nanofiltration and Validation of Prion Removal Method by PrP^{Sc} Antigen Capture Enzyme Immunoassay (EIA) and In Vivo Assay

The scrapie agent used in this example was the hamster 263K strain that was well characterized and widely accepted as a surrogate marker for TSE infectivity. The scrapie preparation used was a 10% hamster brain homogenate that was sonicated, centrifuged at 10,000 rpm for 10 minutes and filtered through a cascade of filters with porosities of 0.45 and 0.22 μm, prior to spiking experiments performed at the following dilutions: 10⁰, 10⁻¹, 10⁻², 10⁻³, 10⁻⁴, 10⁻⁵, 10⁻⁶ and 10⁻⁷.

Bovine blood was obtained from multiple healthy donors or from an individual animal raised under U.S. FDA guidelines. Blood was drawn by puncture of the external jugular vein under aseptic conditions. Approximately 2 liters of blood was obtained from one animal and collected into four 500 mL evacuated, sterile, pyrogen-free bottles containing 75 mL of ACD anticoagulant (The Metrix Company, Dubuque, Iowa 52002, U.S.A.). Blood from different animals was not mixed. The bottles were kept on gel ice in transit to the blood substitute manufacturing facility. The blood was then subjected to separation of red blood cells from leukocytes by LEUKOTRAP and from platelets and plasma by centrifugation. This step reduced the load of non-heme proteins and other substances from which hemoglobin must be ultimately purified. The removal of all leukocytes also removes any viruses associated with these cells such as cytomegalovirus, human immunodeficiency virus and others. Moreover, the complete removal of leukocytes eliminated TSE agents that tended to be present in these cells.

The removal of leukocytes that may carry viruses and TSE agents was performed with a PALL LEUKOTRAP AFFINITY PRION REDUCTION FILTER SYSTEM (Pall Corporation, East Hills, N.Y. 11548, U.S.A.). According to the manufacturer the prion reduction performance for PrP^{Sc} is 2.9±0.7 log. The purification was performed either on whole blood within 8 hours of donation, alternatively on blood held overnight at 4 degrees C., in a volume of 450 mL per filter, at blood temperatures ranging from 4 to 22 degrees C., in accordance with the manufacturer's instructions.

Then, the red blood cells were purified from platelets and plasma by centrifugation at about 170×g at 15 degrees C. for 20 minutes and a series of five washings and five centrifugations with isotonic saline solution (red blood cells:saline, 1:4 vol/vol; 760×g at 4 degrees in a 10 minute cycle) in sterile, pyrogen-free plastic containers (Fenwal Laboratories, Deerfield, Ill. 60015, U.S.A.) using standard blood banking procedures under aseptic conditions.

To confirm the absence of leukocytes and platelets, cell counts were carried out by use of a Coulter cell counter, and the absence of protein in the suspension was verified by routine chemical methods such as a spectrophotometric method.

The extraction of hemoglobin from red blood cells was carried out by hypo-osmotic dialysis—ultrafiltration using a high flow filtration modules with porosity of 0.45 μm. In order to minimize proteolysis during hemoglobin isolation, the procedure was done at 4 degrees C., using slightly hypotonic media (240-260 mOsm kg) and a transmembrane pressure of less than 10 p.s.i. The extracted hemoglobin was filtered through a 0.2 μm filter such as Pall SSUPOR DCF Capsule Filter (Pall Corporation), changed to a carbon-monoxy form by saturation with carbon monoxide, and stored in FENWAL transfer packs at 4 degrees C.

In this example, approximately 500 mL of bovine hemoglobin in a concentration of 60±10 grams per liter in TRIS buffer, pH 6.8±0.2, spiked with a 10% hamster brain homogenate was subjected for nanofiltration using a commercially available high performance hemoconcentrator, HEMOCOR HPH 1400 with optional tubing set (Minntech Corporation). This polysulfone-based hollow fiber dialysis membrane has an effective fiber length of 20.9 cm, a membrane filtration area of 1.31 m², a priming volume of 86 mL and an average molecular weight cutoff of 65 kDa. The filtration was performed with a flow rate of 300 mL/min, a transmembrane pressure of 30 kPa and completed in 2 hours.

The dialysate was collected and concentrated almost to the original level of Hb of 55±8 grams per liter, using a commercially available low flux polysulfone-based dialyzer, OPTIFLUX, with optional tubing set (Fresenius Medical Care, Lexington, Mass. 02420, U.S.A.). This device had a surface area of 1.5 m², a prime volume of 83 mL and an average molecular weight cutoff of 10 kDa.

The procedure was completed in approximately 1 hour, and the concentrated product was subjected, along with the pre-dialysis samples for measurement of the prion protein levels by BSE-SCRAPIE ANTIGEN TEST EIA KIT (IDEXX Laboratories, Inc., Westbrook, Me. 04092, U.S.A.) that recognizes PrP^{Sc}, according to the manufacturer. Alternatively, after treatment with proteases, the sample was run using a SPI-BIO EIA kit (Cayman Chemical Co., Ann Arbor, Mich. 48108, U.S.A.) that employs two antibodies that were raised against a preparation of denatured scrapie associated fibril agents (SAFs) from infected hamster brain, according to the manufacturer's instructions.

All experiments were done in triplicate and clearance of PrP^{Sc} was expressed by calculation of the log reduction factor (RF) using the equation: RT=log 10 (sample starting volume×initial PrP^{Sc} concentration)/(sample volume after filtration×final PrP^{Sc} concentration). Results indicated that HEMOCOR HPH 1400 filtered more than 90% of hemoglobin after 2 hours, at a ratio of hemoglobin to hollow fiber surface area of 400 mL per m² and as indicated in Table 1, the nanofiltration was able to reduce the PrP^{Sc} level by an average 3.47±0.14 logs.

TABLE 1

| | LOG₁₀ REDUCTION FACTOR |
|---|---|
| RUN No. 1 | 3.41 |
| RUN No. 2 | 3.63 |
| RUN No. 3 | 3.37 |
| MINIMUM: | 3.37 |
| MAXIMUM: | 3.63 |
| RANGE: | 0.26 |
| MEDIAN: | 3.41 |
| MEAN: | 3.47 |
| STANDARD ERROR: | 0.08 |
| VARIANCE: | 0.02 |
| STANDARD DEVIATION: | 0.14 |
| COEFFICIENT OF VARIATION: | 4.03 |

The filtrates, which were also evaluated by in vivo assay were: (1) the bovine hemoglobin solution spiked with scrapie agent and not subjected to the nanofiltration processand (2) the bovine hemoglobin solution spiked with PrP^{Sc} and subjected to nanofiltration, both samples were evaluated at the following dilutions: 10⁰, 10⁻¹, 10⁻², 10⁻³, 10⁻⁴, 10⁻⁵, 10⁻⁶ and 10⁻⁷.

The in vivo assay for scrapie infectivity involved intracerebral (i.e.) inoculation of hamsters (weanlings approximately 6-8 weeks of age) with an aliquot of a solution of interest. Five hamsters were assigned to each dilution group of spiked unpurified and spiked purified hemoglobin solutions (5 animals per dilution and seven dilutions per titration). Control hamsters were inoculated with hemoglobin alone. The animals were observed daily for 200 days and monitored for typical clinical signs of scrapie infection (ataxia, chronic wasting and neurological characteristics such as circular wandering) and survival rates. The 200 day observation period was chosen based on the indication that transmission of bovine prions to transgenic mice exhibit an incubation time of approximately 200 days. Thus, after 200 days, all surviving animals were sacrificed by an anesthesia overdose and their brains were examined by electron microscopy for characteristic tubuli of scrapie infection, scrapie associated fibril agent. The brains of dead animals and those terminated due to clinical signs of scrapie infection were also examined by electron microscopy for SAF. The survival and SAF positive rates are presented in Table 2.

The results suggest that the spiked unpurified bovine hemoglobin preparation has a scrapie infectivity titer of approximately 10⁵/mL. After nanofiltration the reduction in scrapie infectivity of approximately 10^{3.5} was achieved. These results are consistent with the results obtained by ELIA studies. Thus, nanofiltration alone was unable to fully eliminate the PrP^{Sc} infectivity, and at dilutions of 10⁰ and 10⁻¹ some animals did not survive. Further, the results suggest that nanofiltration, even through hollow fibers with pore size of about 65 kDa, cannot serve as an independent method for complete PrP^{Sc} clearance from hemoglobin solution when a scrapie infectivity titer is about 10⁵/mL.

TABLE 2

| | NO. OF ANIMALS DEAD/SCRAPIE CONSISTENT | | | | | | | |
|---|---|---|---|---|---|---|---|---|
| | PATHOLOGY AT DIFFERENT DILUTIONS | | | | | | | |
| | DILUTION | | | | | | | |
| SAMPLE | 10⁰ | 10⁻¹ | 10⁻² | 10⁻³ | 10⁻⁴ | 10⁻⁵ | 10⁻⁶ | 10⁻⁷ |
| PRE- | 5/5 | 5/5 | 4/5 | 5/5 | 1/3 | 0/1 | 0/0 | 0/0 |
| FILTRATION | | | | | | | | |
| POST- | 3/5 | 1/3 | 0/0 | 0/0 | 0/0 | 0/0 | 0/0 | 0/0 |
| FILTRATION | | | | | | | | |
| UNSPIKED | 0/0 | 0/0 | 0/0 | 0/0 | 0/0 | 0/0 | 0/0 | 0/0 |
| HEMOGLOBIN | | | | | | | | |
| CONTROL | | | | | | | | |

### Example Two

### Purification of Bovine Hemoglobin Solution by Anion Exchange Membrane Chromotography and Validation of Prion Removal Method by PrP^{Sc} Antigen Capture Enzyme Immunoassay (EIA)

The scrapie agent also used in this example was the hamster 263K strain. The scrapie preparation used was a 10% hamster brain homogenate that was sonicated, centrifuged at 10,000 rpm for 10 minutes and filtered through a cascade of filters with porosities of 0.45 and 0.22 μm, prior to spiking experiments performed at the following dilutions: 10⁰, 10⁻¹, 10⁻², 10⁻³, 10⁻⁴, 10⁻⁵, 10⁻⁶ and 10⁻⁷.

In this example, 100 mL of bovine hemoglobin solution, prepared as in Example 1, in a concentration of 60±10 grams per liter in TRIS buffer, pH 6.8±2, spiked with a 10% hamster brain homogenate, was subjected for anion exchange membrane chromatography using a commercially available Pall ACRODISC Unit with MUSTANG Q MEMBRANE (Pall Corporation, Ann Arbor, Mich. 48103-9019, U.S.A.).

MUSTANG Q polyethersulfone membrane, with 0.8 μm porosity, is a strong anion exchanger that effectively binds plasmid DNA, negatively-charged proteins, and viral particles. The chromatography was performed using one disposable Pall ACRODISC unit per 20 mL of hemoglobin. Before chromatography, the ACRODISC unit was preconditioned with 4 mL 1 M NaOH followed by 4 mL of 1 M NaCl, and equilibrated with 20 mM TRIS buffer, pH 6.8+0.2. Spiked hemoglobin solution (pH 6.8±0.2) in the carbon-monoxy form, was subjected to chromatographic separation at a flow of 4 mL/min. At a pH of 6.8, hemoglobin is without charge. The elimination of the electric charge of hemoglobin is intended to prevent its binding to this strong anion exchange membrane equilibrated with 20 mM TRIS buffer with a pH of 6.80.2. This chromatography method is also intended not to affect the binding of DNA and viral particles to the membrane. After 5 chromatographic runs, using separate ACRODISC units, the collected fractions were pooled together and the final volume determined.

The pre- and post-chromatography samples in the following dilutions: 10⁰, 10⁻¹, 10⁻², 10⁻³, 10⁻⁴, 10⁻⁵, 10⁻⁶ and 10⁻⁷, were subjected for measurement of the prion protein levels by BSE-SCRAPIE ANTIGEN TEST EIA KIT (IDEXX Laboratories, Inc., Westbrook, Me. 04092, U.S.A.) that recognizes PrP^{Sc}, according to the manufacturer's instructions. Additionally, after treatment with proteases, the samples were run by SPI-BIO EIA kit (Cayman Chemical Co., Ann Arbor, Mich. 48108, U.S.A.) that employs two antibodies that were raised against a preparation of denatured SAFs from infected hamster brain, according to the manufacturer's instructions.

All experiments were done in triplicate. Clearance of PrP^{Sc} was expressed by calculation of the log reduction factor (RF) using the equation: RT=log 10 (sample starting volume×initial PrP^{Sc} concentration)/(sample volume after filtration×final PrP^{Sc} concentration). The results indicated that the samples contacted with the MUSTANG Q MEMBRANE anion exchanger did not exhibit a decrease in the level of hemoglobin, when the ratio of hemoglobin to membrane surface area of the exchanger was approximately 5 mL per cm². However, as indicated in Table 3, anion exchange membrane chromatography with MUSTANG Q was able to reduce the PrP^{Sc} level in the sample by 4.01±0.24 logs.

TABLE 3

| | LOG₁₀ REDUCTION FACTOR |
|---|---|
| RUN No. 1 | 3.81 |
| RUN No. 2 | 3.94 |
| RUN No. 3 | 4.27 |
| MINIMUM: | 3.81 |
| MAXIMUM: | 4.27 |
| RANGE: | 0.46 |
| MEDIAN: | 3.94 |
| MEAN: | 4.01 |
| STANDARD ERROR: | 0.14 |
| VARIANCE: | 0.06 |
| STANDARD DEVIATION: | 0.24 |
| COEFFICIENT OF VARIATION: | 5.92 |

### Example Three

### Purification of Bovine Hemoglobin Solution by Hydrophobic Solvent and Validation of Prion Removal Method by PrP^{Sc} Antigen Capture Enzyme Immunoassay (EIA)

The scrapie agent also used in this example was the hamster 263K strain. The scrapie preparation used was a 10% hamster brain homogenate that was sonicated, centrifuged at 10,000 rpm for 10 minutes and filtered through a cascade of filters with porosities of 0.45 and 0.22 μm, prior to spiking experiments performed at the following dilutions: 10⁰, 10⁻¹, 10⁻², 10⁻³, 10⁻⁴, 10⁻⁵, 10⁻⁶ and 10⁻⁷.

In this example, 200 mL of bovine hemoglobin solution in carbon-monoxy form, prepared as in the Example 1, in a concentration of 60±10 grams per liter in TRIS buffer, pH 8.0±2, spiked with a 10% hamster brain homogenate, was subjected to hydrophobic solvent treatment with chloroform (HPLC Grade, Fisher Scientific).

A series of three treatments with chloroform followed by centrifugation steps were carried out using a Sorvall centrifuge (Model RC5C with SS-34 rotor), in the following manner: (1) hemoglobin mixed with chloroform at a ratio of 15 to 1 (vol/vol) was vortexed for 15 minutes and centrifuged at 760×g and 4 degrees C., for 30 minutes; (2) the supernatants were passed into a second series of tubes, mixed with chloroform at a ratio of 16 to 1 (vol/vol), vortexed for 10 minutes and centrifuged at 1,600×g and 4 degrees C., for 15 minutes, and at 3,800×g for 15 minutes; (3) the supernatants were transferred into a third series of tubes and centrifuged without chloroform at 48,400×g and 4 degrees C., for 90 minutes. After the third centrifugation, the hemoglobin solution was subjected to removal of remaining traces of chloroform by flushing with nitrogen gas followed by carbon monoxide to assure its full conversion to carbon-monoxy form.

The pre- and post-chloroform treated samples in the following dilutions: 10⁰, 10⁻¹, 10⁻², 10⁻³, 10⁻⁴, 10⁻⁵, 10⁻⁶ and 10⁻⁷were subjected for measurement of the prion protein levels by BSE-SCRAPIE ANTIGEN TEST EIA KIT (IDEXX Laboratories, Inc., Westbrook, Me. 04092, U.S.A.) that recognizes PrP^{Sc}, according to the manufacturer's instructions. Additionally, after treatment with proteases, the samples were run by SPI-BIO EIA kit (Cayman Chemical Co., Ann Arbor, Mich. 48108, U.S.A.) that employs two antibodies that were raised against a preparation of denatured SAFs from infected hamster brain, according to the manufacturer's instructions.

All experiments were done in triplicate. Clearance of PrP^{Sc} was expressed by calculation of the log reduction factor (RF) using the equation: RT=log 10 (sample starting volume×initial PrP^{Sc} concentration)/(sample volume after filtration×final PrP^{Sc} concentration). As indicated in Table 4, a treatment with chloroform reduced the PrP^{Sc} level by 1.15±0.14 logs. This data suggests that chloroform treatment can be considered as an inactivation step with respect to purification of hemoglobin solutions from PrP^{Sc}.

TABLE 4

| | LOG₁₀ REDUCTION FACTOR |
|---|---|
| RUN No. 1 | 1.15 |
| RUN No. 2 | 1.03 |
| RUN No. 3 | 0.87 |
| MINIMUM: | 0.87 |
| MAXIMUM: | 1.15 |
| RANGE: | 0.28 |
| MEDIAN: | 1.03 |
| MEAN: | 1.02 |
| STANDARD ERROR: | 0.08 |
| VARIANCE: | 0.02 |
| STANDARD DEVIATION: | 0.14 |
| COEFFICIENT OF VARIATION: | 13.82 |

### Example Four

### Purification of Bovine Hemoglobin Solution by Nanofiltration, Anion Exchange Membrane Chromatography and Hydrophobic Solvent and Validation of Prion Removal Method by In Vivo Assay

The scrapie agent also used in this example was the hamster 263K strain. The scrapie preparation used was a 10% hamster brain homogenate that was sonicated, centrifuged at 10,000 rpm for 10 minutes and filtered through a cascade of filters with porosities of 0.45 and 0.22 μm, prior to spiking experiments performed at the following dilutions: 10⁰, 10⁻¹, 10⁻², 10⁻³, 10⁻⁴, 10⁻⁵, 10⁻⁶ and 10⁻⁷.

The solutions evaluated by in vivo assay were: (1) the bovine hemoglobin solution spiked with scrapie agent and not subjected to the prion purification process, in the following dilutions: 10⁰, 10⁻¹, 10⁻², 10⁻³, 10⁻⁴, 10⁻⁵, 10⁻⁶ and 10⁻⁷and (2) the bovine hemoglobin solution spiked with scrapie agent and subjected to the cascade prion purification process based on nanofiltration, anion exchange membrane chromatography and hydrophobic treatment, in the following dilutions: 10⁰, 10⁻¹, 10⁻², 10⁻³, 10⁻⁴, 10⁻⁵, 10⁻⁶ and 10⁻⁷. The starting material for this example was bovine hemoglobin solution, in carbon-monoxy form, and was prepared as in Example 1, and spiked as in described previously.

The TSE purification process combined: (1) nanofiltration, (2) anion exchange membrane chromatography and (3) hydrophobic solvent treatment with chloroform, as described in Examples 1, 2 and 3, respectively. To maintain low absorption of hemoglobin to nanofiltration and anionic membrane exchange devices, hemoglobin was dissolved in a buffer system that eliminated its charge and therefore its electrostatic interaction. Such a buffer system is described in Examples 1 and 2. Additionally, in order to protect heme against oxidation, the heme oxygen was completely replaced with carbon monoxide, forming carbon-monoxy hemoglobin, which is highly resistant to oxidative challenge. Any changes in sample volume were corrected for dilution by estimating hemoglobin concentration. The average hemoglobin concentrations in pre-purified samples were approximately 60±10 grams per liter and after purification, were approximately 55±8 grams per liter.

The in vivo assay for scrapie infectivity involved intracerebral (i.c.) inoculation of hamsters (weanlings approximately 6-8 weeks of age) with an aliquot of a solution of interest. Five hamsters were assigned to each dilution group of spiked unpurified and spiked purified hemoglobin solutions (5 animals per dilution and seven dilutions per titration). Control hamsters were inoculated with hemoglobin alone. The animals were observed daily for 200 days and monitored for typical clinical signs of scrapie infection (ataxia, chronic wasting and neurological characteristics such as circular wandering) and survival rates. After 200 days, all surviving animals were sacrificed by an anesthesia overdose and their brains were examined by electron microscopy for characteristic tubuli of scrapie infection (scrapie associated fibril agent—SAF). The brains of dead animals and those terminated due to clinical signs of scrapie infection were also examined by electron microscopy for SAF. The survival and SAF positive rates are presented in Table 5.

Results suggest that the spiked unpurified bovine hemoglobin preparation has a scrapie infectivity titer of approximately 10⁵/mL. After this multi-step purification procedure of the scrapie spiked bovine hemoglobin samples, no scrapie infectivity was detectable. Inoculation of animals with hemoglobin alone did not result in any observed clinical and morphological changes.

These data suggest that purification of bovine hemoglobin solution from TSE agent by sequential stepwise use of nanofiltration, anion exchange membrane chromatography and hydrophobic solvent treatment can effectively eliminate scrapie infectivity.

This multi-step purification procedure of bovine hemoglobin from PrP^{Sc} may be considered as orthogonal, since it contains elements of removal (nanofiltration and anion exchange membrane chromatography) and inactivation (hydrophobic solvent) of the TSE agent.

TABLE 5

| | NO. OF ANIMALS DEAD/SCRAPIE CONSISTENT | | | | | | | |
|---|---|---|---|---|---|---|---|---|
| | PATHOLOGY AT DIFFERENT DILUTIONS | | | | | | | |
| | DILUTION | | | | | | | |
| SAMPLE | 10⁰ | 10⁻¹ | 10⁻² | 10⁻³ | 10⁻⁴ | 10⁻⁵ | 10⁻⁶ | 10⁻⁷ |
| PRE- | 5/5 | 5/5 | 4/5 | 5/5 | 1/2 | 0/1 | 0/0 | 0/0 |
| PURIFICATION | | | | | | | | |
| POST- | 0/0 | 0/0 | 0/0 | 0/0 | 0/0 | 0/0 | 0/0 | 0/0 |
| PURIFICATION | | | | | | | | |
| UNSPIKED | 0/0 | 0/0 | 0/0 | 0/0 | 0/0 | 0/0 | 0/0 | 0/0 |
| HEMOGLOBIN | | | | | | | | |
| CONTROL | | | | | | | | |

While embodiments have been shown and described, modifications thereof can be made by one skilled in the art without departing from the spirit and teachings of the invention. The embodiments described herein are exemplary only, and are not intended to be limiting. Many variations and modifications are possible and are within the scope of the disclosure. Where numerical ranges or limitations are expressly stated, such express ranges or limitations should be understood to include iterative ranges or limitations of like magnitude falling within the expressly stated ranges or limitations (e.g., from about 1 to about 10 includes, 2, 3, 4, etc.; greater than 0.10 includes 0.11, 0.12, 0.13, etc.). Use of the term “optionally” with respect to any element of a claim is intended to mean that the subject element is required, or alternatively, is not required. Both alternatives are intended to be within the scope of the claim. Use of broader terms such as comprises, includes, having, etc. should be understood to provide support for narrower terms such as consisting of, consisting essentially of, comprised substantially of, etc.

Accordingly, the scope of protection is not limited by the description set out above but is only limited by the claims which follow, that scope including all equivalents of the subject matter of the claims. Each and every claim is incorporated into the specification as an embodiment of the present invention. Thus, the claims are a further description and are an addition to the embodiments of the present invention. The discussion of a reference herein is not an admission that it is prior art to the present invention, especially any reference that may have a publication date after the priority date of this application. The disclosures of all patents, patent applications, and publications cited herein are hereby incorporated by reference, to the extent that they provide exemplary, procedural or other details supplementary to those set forth herein.

1. A method comprising:
contacting a biological fluid comprising hemoglobin and at least one pathogenic agent with a first filter and generating a first filtrate;
contacting the first filtrate with a nanofiltration device and generating a second filtrate;
contacting the second filtrate with a chromatographic material and isolating an eluted fraction;
contacting the eluted fraction with a hydrophobic solvent and generating a hydrophobic and a hydrophilic phase; and
isolating the hydrophilic phase, wherein the biological fluids comprise components of interest of equal to or less than about 65 kDa.

2. The method of claim 1 further comprising saturating the biological fluid comprising hemoglobin with carbon monoxide prior to contact with the first filter.

3. The method of claim 1 wherein the biological fluid comprises human derived hemoglobin, animal-derived hemoglobin, or combinations thereof.

4. The method of claim 1 wherein the pathogenic agent comprises a proteineceous prion, a transmission spongiform encepahalpthy agent, or combinations thereof.

5. The method of claim 1 wherein the first filter comprises a high flow affinity prion reduction filter.

6. The method of claim 5 wherein the filter has a flow rate of from about 500 ml to about 1000 ml in equal to or less than about 25 minutes.

7. The method of claim 1 wherein the amount of pathogenic agent in the first filtrate is reduced by equal to or greater than about 1 log when compared to the amount of pathogenic agent in the biological fluid.

8. The method of claim 1 wherein the nanofiltration device comprises a hollow fiber filter or a disc.

9. The method of claim 8 wherein the nanofiltration device has a molecular weight cutoff of about 65 kDa.

10. The method of claim 1 wherein the amount of pathogenic agent in the second filtrate is reduced by equal to or greater than about 1 log when compared to the amount of pathogenic agent in the first filtrate.

11. The method of claim 1 wherein the chromatographic material comprises a strong anion exchanger.

12. The method of claim 1 wherein the amount of pathogenic agent in the eluted fraction is reduced by equal to or greater than about 1 log when compared to the amount of pathogenic agent in the second filtrate.

13. The method of claim 1 wherein the hydrophobic solvent comprises chloroform, toluene, or combinations thereof.

14. The method of claim 1 wherein the amount of pathogenic agent in the hydrophilic phase is reduced by equal to or greater than about 5 logs when compared to the amount of pathogenic agent in the biological fluid.

15. The method of claim 1 wherein the hydrophilic phase is not infective.

16. The method of claim 1 further comprising determining the amount of pathogenic agent.

17. The method of claim 16 wherein determination of the amount of pathogenic agent in the composition is carried out by Western blot analysis, ELISA, animal infectivity assays, or combinations thereof.

18. A method comprising:
contacting a biological fluid comprising high molecular weight components and at least one pathogenic agent with a first filter and generating a first filtrate;
contacting the first filtrate with a hydrophilic membrane and generating a second filtrate;
contacting the second filtrate with a chromatographic material and isolating an eluted fraction;
contacting the eluted fraction with a hydrophobic solvent and generating a hydrophobic and a hydrophilic phase; and
isolating the hydrophilic phase, wherein the high molecular weight components have molecular weights greater than about 65 kDa.

19. The method of claim 18 wherein the hydrophilic membrane comprises polyvinylidene fluoride.

20. A method comprising:
subjecting a biological fluid comprising hemoglobin and at least one pathogenic agent to at least two filtration steps and thereby reducing the amount of pathogenic agent associated with the biological fluid.

21. The method of claim 20 wherein the pathogenic agent comprises a transmission spongiform encephalapthy agent and the reduction in the amount of the agent is equal to or greater than about 5 logs.

22. A method comprising:
removing transmissible spongiform encephalopathy agents in a hemoglobin solution of human and/or animal origin by subjecting the hemoglobin solution to an orthogonal separation methodology comprising a plurality of filtration steps.
